(19) United States
(12) Patent Application Publication
(10) Pub. No.: US 2005/0178416 A1
(43) Pub. Date: Aug. 18, 2005

(54) Lower limb-support ambulatory device

(76) Inventor: Tony Leroy Owens, Portland, OR (US)

Correspondence Address: IDEA ADVOCATES LAW GROUP LLC, 322 NW 5TH AVE #310, PORTLAND, OR, 97209, US

(21) Appl. No.: 11/041,502

(22) Filed: Jan. 24, 2005

Related U.S. Application Data
(60) Provisional application No. 60/543,561, filed on Feb. 12, 2004.

(51) Int. Cl. A61F 5/00; A61H 3/02

(52) U.S. Cl. 135/69

(57) ABSTRACT

A lower limb, e.g. bent knee, supporting device includes a lower limb supporting brace including at an upper extreme thereof and aligned with a long axis thereof a cushioned limb support member; an elongate vertical stabilizing handle or member for stabilizing the brace, the handle being securely grippable by a user without use of the armpit; the brace including at a lower extreme a tip configured to frictionally engage an ambulatory surface; the brace and the handle being affixed to one another along parallel extents thereof. The device includes one or more grippable collars aligned along a long vertical axis of the handle. The handle extends to a height intermediate the user's waist and armpit so that the upper handle can be squeezed between the user's arm and torso, thus freeing both hands for other uses. The height of the limb support member is adjustable so that its concave upper surface can cup a bent knee of a user with the lower leg extending behind. The device is lightweight and the handle provides significant leverage in positioning, aligning and securing the device to the lower limb.

### RELATED APPLICATIONS

The present application claims priority to U.S. Provisional Patent Application Ser. No. 60/543,561, entitled KNEE SUPPORT CRUTCH and filed Feb. 12, 2004, the disclosure of which is incorporated herein in its entirety by this reference.

### BACKGROUND OF THE INVENTION

This invention relates generally to the field of apparatus for a cane, walker or crutches used by the physically injured or disabled as an aid to ambulating (walking), the cane usually being held in one hand, the walker usually being held in two hands and the crutches being held one in either hand and being thrust under either armpit.

### SUMMARY OF THE INVENTION

A lower limb, e.g. bent knee, supporting device includes a lower limb supporting brace including at an upper extreme thereof and aligned with a long axis thereof a cushioned limb support member; an elongate vertical stabilizing handle or member for stabilizing the brace, the handle being securely grippable by a user without use of the armpit; the brace including at a lower extreme a tip configured to frictionally engage an ambulatory surface; the brace and the handle being affixed to one another along parallel extents thereof. The device includes one or more grippable collars aligned along a long vertical axis of the handle. The handle extends to a height intermediate the user's waist and armpit so that the upper handle can be squeezed between the user's arm and torso, thus freeing both hands for other uses. The height of the limb support member is adjustable so that its concave upper surface can cup a bent knee of a user with the lower leg extending behind. The device is lightweight and the handle provides significant leverage in positioning, aligning and securing the device to the lower limb.

### BRIEF DESCRIPTION OF THE DRAWINGS

FIGS. 1A and 1B are front isometric views of the knee support ambulatory device in use by a user who is standing on, exercising, ambulating (walking) or resting his injured leg, FIG. 1A showing the device in one-handed operation and FIG. 1B showing the device in no-handed operation.

FIG. 2. is a front elevation of the knee support ambulatory device according to the present invention.

FIG. 3. is a side elevation of the knee support ambulatory device according to the present invention.

FIG. 4 is an isometric view of the knee support ambulatory device in use by a seated user who is sitting and resting his injured leg.

### DETAILED DESCRIPTION OF THE PREFERRED EMBODIMENTS

The invention in accordance with a preferred embodiment involves a singular lower limb-support, e.g. a bent-knee support, device that is somewhat similar to a conventional crutch but that allows one or both hands to be free to carry or use objects. Those of skill in the art will appreciate that conventional crutches are used in bilateral pairs, with the top curved surfaces thrust into either armpit by the user's two-handed grip on the crutch pairs' handles.

Thus, the advantages of the invented device over conventional crutches include a) avoiding nerve damage from pressure to the armpit, wrist and hand areas caused by placing unnecessary weight or strain on these areas of the user's body; and b) avoiding atrophy of the upper half of the injured leg by continually exercising the same during use of the invented device.

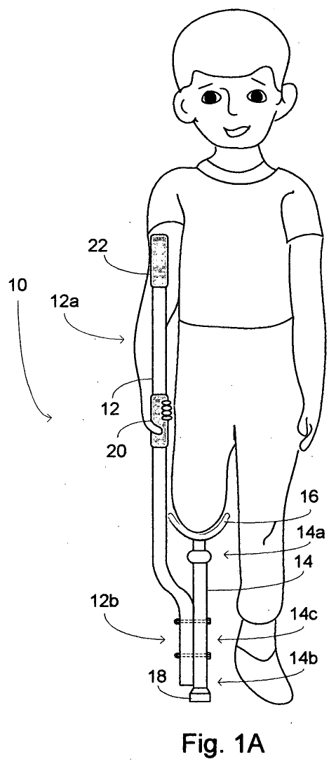

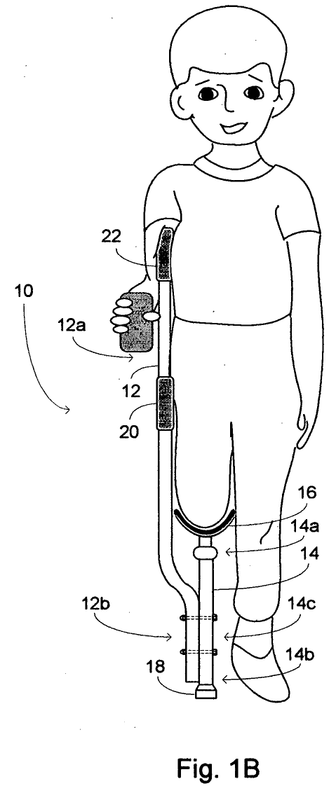

FIGS. 1A, 1B, 2, 3 and 4 illustrate a knee support ambulatory device made in accordance with one embodiment of the invention that has been discovered to realize the advantages listed above.

FIGS. 1A and 1B illustrate the invented lower limb-support ambulatory device 10 in a front isometric view illustrating a user who may be standing, exercising, ambulating (walking) or resting, FIG. 1A showing the device in one-handed operation and FIG. 1B showing the device in no-handed operation. FIGS. 1A and 1B are illustrative of device 10 in one embodiment of the invention and its use in accordance with one method of using the invention. Those of skill in the art will appreciate that alternative embodiments of the invention, as well as alternative methods of using the invention, are contemplated as being within the spirit and scope of the invention. For example, the invented device can be used to support the lower limb extremity of an amputee during a transitional phase of rehabilitation after amputation of a lower limb and before fitting of the lower limb with a prosthetic.

Invented device 10 may be understood to include a vertical member 12 configured to be grippable by a user in an upper region 12a thereof; and a lower limb support brace 14 affixed to vertical member 12, lower limb support brace 14 being configured at an upper extreme 14a thereof with a lower limb support member 16 to support, for example, a bent knee of the user. Lower limb support brace 14 includes at a lower extreme 14b thereof a tip 18 configured to frictionally engage an ambulation surface on which the user ambulates while gripping the vertical member with the bent knee, for example, reposing on the support member.

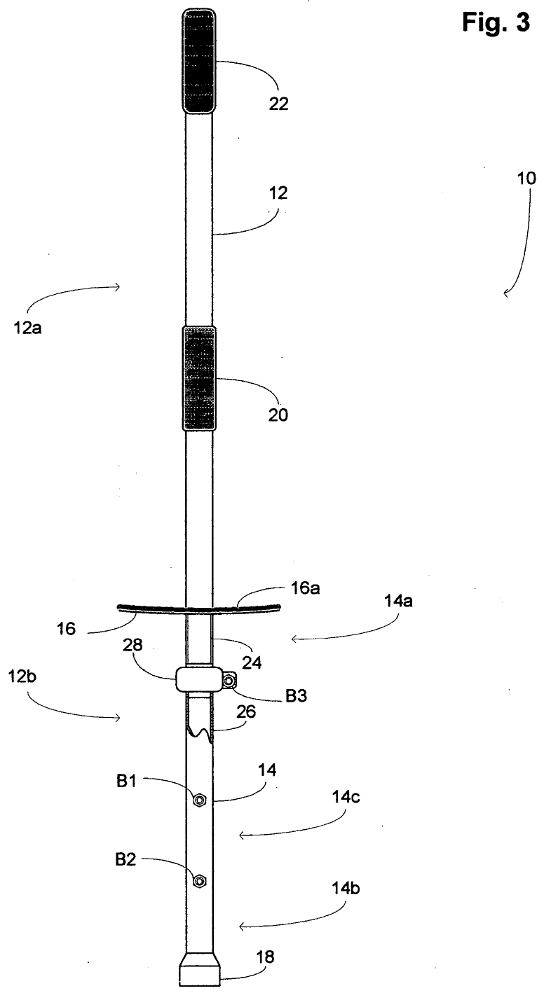

As may be seen from FIGS. 1A and 1Bs, vertical member 12 is elongate and includes at least one cushioned gripping collar or hand grip 20 aligned with its long vertical axis and optionally an additional cushioned gripping collar or hand grip 22 similarly aligned and positioned at an extreme upper end of vertical member 12. Also as may be seen from FIGS. 1A and 1B, lower limb support brace 14 is positioned laterally inwardly from vertical member 12 when the device is in use. Those of skill in the art will appreciate that this is true whether device 10 is used on the right or left side of the body to support a right or left lower limb such as a bent knee. As will be seen from FIG. 3, invented device 10 is substantially symmetric front to back, so that it is readily reversed left to right for use on either side.

It may be seen from FIGS. 1A and 1B that vertical member 12 is dimensioned relative the user such that its upper extreme (where optional gripping collar 22 is positioned) reaches a height on the user approximately intermediate his waist and armpit. This renders invented device 10 extremely easily manipulated by vertical member 12 to lift, pivot or otherwise stabilize lower limb brace 14. The mechanical advantage of leverage provides ease of use and secure but temporary fixation of the lower limb, e.g. the bent knee, within the lower limb support brace. Thus, the elongate vertical member effectively acts as a handle that positions, aligns and secures the support brace relative to the lower limb.

Briefly comparing and contrasting FIGS. 1A and 1B, it may be seen that lower grip 20 permits device 10 easily to be operated with one hand while upper grip 22 alternatively permits device 10 easily to be operated, at least over short distances, with no hand on lower grip 20. In other words, FIG. 1A illustrates one-handed operation of device 10 and FIG. 1B illustrates no-handed operation of device 10. Thus, the user of device 10 in FIG. 1A grips the device lightly in one hand and accordingly has the other hand free but the user of identical device 10 in FIG. 1B squeezes the device lightly between his upper arm and torso and accordingly has both hands free, as indicated by the mug he carries in his right hand. The novel combination of the relatively vertical handle member (12) extending to an elevation approximately intermediate the user's waist and armpit, including the two or more grips (20, 22) spaced apart therealong, and aligned axially therewith, the lower limb support mechanism (14) and component parts of lightweight and preferably round, hollow, tubular material, renders possible such convenience. The ability to use a lower limb-support ambulatory device in this way represents an important advantage of the invention over conventional crutches.

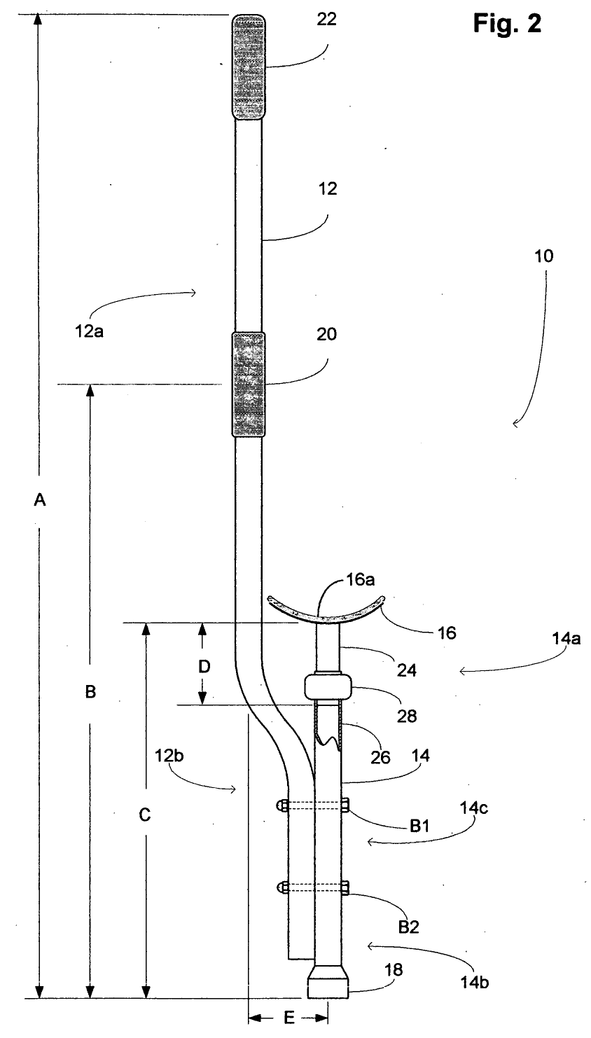

In accordance with one embodiment of the invention, as will be seen from FIGS. 2 and 3, lower limb support member 16 preferably is height-adjustable relative to tip 18. Also, it will be understood that, in accordance with one embodiment of the invention, gripping collar 20 is slidably but securely height-adjustable along vertical member 12. (This is possible by use of a collar materially and structurally similar to a handlebar grip on a bicycle's steering wheel, as is known, whereby the collar has an interference fit with the hollow tubing sufficient to securely hold it in place against inadvertent sliding but insufficient to immobilize it against intentional sliding relative to the tubing.) Thus, invented device 10 accommodates a range of user sizes and preferences.

Referring collectively to FIGS. 2 and 3, a preferred embodiment of invented device 10 will be described in structural and material detail. Those of skill in the art will appreciate that the detailed material and structural embodiment described and illustrated herein is intended to illustrate and not to limit the scope of the invention. For example, it will be seen that preferably vertical member 12 (also referred to herein as a stabilizing handle for stabilizing lower limb supporting brace 14) and lower limb support brace 14 are lengths of hollow aluminum tubing of circular cross section that are, along overlapping, parallel extents thereof, bolted together. Forming the major component parts of device 10 of hollow aluminum tubing renders the device lightweight, e.g. approximately 3 pounds overall, which helps achieve one of the advantages of the invention—easy employment and deployment in terms of grip, positioning, alignment, fixation and maneuverability. However, those of skill in the art will appreciate that the member and brace can be made of any suitable material that is lightweight and durable, e.g. titanium, graphite, fiberglass or plastic (e.g. PVC) or their combination. Those of skill in the art also will appreciate that the component parts can be differently affixed together or integrally molded or otherwise formed to provide an equivalent structure featuring lightweight but durable functionality, all within the spirit and scope of the invention.

In accordance with one embodiment of the invention, vertical member 12 includes a length of hollow, circularly cross-sectioned tubing that is generally S-shaped, as illustrated in FIG. 2. Also in accordance with one embodiment of the invention, lower limb support brace 14 includes a length of hollow, circularly cross-sectional tubing that is generally straight. Vertical member 12 is configured to be placed near the upper right side or left side (when reversed front to back) of the user's torso, extending to a height A that is approximately intermediate the user's waist and armpit and extending downwardly and stepping laterally inwardly so that it generally parallels and conforms with the torso and leg of the user, as shown in FIG. 1. Positioned in an upper region 12a of vertical member 12 and concentrically, axially aligned therewith is a collar-like grip 20 that is adjustably fixed at approximately the height B of the user's crotch so that the user's hands at rest with the user in a standing position are able to reach grip 20. Lower limb support brace 14 is positioned laterally inwardly from vertical member 12 and extends vertically from the ambulation surface to mount lower limb support member 16 to a height C thereabove. Preferably, height C of lower limb support member 16 is adjustable over a range D. Those of skill in the art will appreciate that the lateral, inward step represented by S-shaped vertical member 12 and the spacing between vertical member 12 and lower limb support brace 14 is of dimension E.

Those of skill in the art will appreciate that the dimensions represented by reference designators A, B, C, D and E in FIG. 2 are determined in part by the user's size and preference. Typically, for an adult user, the overall height A of device 10 is approximately four feet or forty-eight inches; the height B of hand grip 10 is approximately thirty inches; the height C of lower limb support member 16 is approximately eighteen inches; the range D of height adjustability of lower limb support member 16 is approximately five inches; and the dimension of the overall lateral inward step between vertical member 12 and lower limb support member 16 is approximately five inches. Any suitable dimensions are contemplated as being within the spirit and scope of the invention.

Different user heights, upper leg extents, torso shapes (body conformation) and preferences, within the spirit and scope of the invention, may make alternative dimensions more suitable to achieving the goals and advantages of the invention. For a user who is over six feet tall, e.g. 6′5″, height A of device 10 might be fifty-one or fifty-two inches or more, whereas for a user who is less than five feet tall, e.g. a child, height A of device 10 might be closer to 36″.

It may be seen from FIGS. 2 and 3 that vertical member 12 in accordance with one embodiment of the invention in lower region 12b thereof is bolted (e.g. by two bolt/nut pairs B1, B2 extending through pairs of aligned holes, as illustrated best in FIG. 2) and corresponding nuts to a central region 14c of lower limb support brace 14. Alternative means of affixing or joining vertical member 12 and lower limb support brace 14 are contemplated as being within the spirit and scope of the invention. For example, they may be welded or glued or integrally formed as a single piece. Alternatively, a lower extreme of handle 12 may simply abut and join brace 14, as by welding or integrally molding both parts together as one, rather than having lower region 12b that runs in parallel therewith and is affixed thereto in region 14c. Thus, it is within the spirit and scope of the invention to construct invented device 10 with more or fewer pieces affixed or formed in different ways.

Centrally mounted in axial alignment at an upper extreme of lower limb support brace 14 is lower limb support member 16. Those of skill in the art will appreciate that lower limb support member 16 provides a padded rest or stop for the lower limb of the user. Thus, it is preferably concave and generally semi-circular in shape from side to side and configured to receive and support, e.g. cup, a user's knee, as illustrated in the front elevational view of FIG. 2. From FIG. 2, it may be seen to be only slightly concave and only slightly generally semi-circular in shape from front to back, as illustrated in the side elevational view of FIG. 3. Those of skill will appreciate that this difference in curvature of lower limb support member 16 effectively cups the lower limb and seats it properly therein to prevent lateral movement while it also cradles the lower limb, e.g. a bent knee of the user, and supports it properly thereon to accommodate fore and aft movement. Those of skill also will appreciate that lower limb support member 16 preferably conforms somewhat to the anatomy of the user's lower limb. Nevertheless, within the spirit and scope of the invention, it may be a simple shelf having no concavity or depression along either orthogonal axis.

In accordance with one embodiment of the invention, an upper, limb-confronting surface 16a of lower limb support member 16 is cushioned or padded to increase the comfort of the user of invented device 10. Such a cushion or pad may, within the spirit and scope of the invention, take the form of an adhered or integral laminar layer of conformable material such as polystyrene foam or the like. Any suitable surface material, of any desired durability and conformability, is contemplated as being within the spirit and scope of the invention.

As is evident from FIG. 1, a user of the invented device places the lower limb, e.g. the bent knee, of the injured leg into the padded rest and places his or her weight onto the padded rest. The lower half of the user's leg, for example, can protrude to the rear. This allows the user to spread the weight of his or her body evenly between the non-injured leg and the lower limb, e.g. the bent knee, of the injured leg. The user squeezes hand grip 20 or pressure grip 22 with the hand or against the body, for example, to stabilize the lower limb support member 16 and, to the extent needed, ever so slightly to elevate the lower limb support member 16 so that the lower limb remains securely in place within padded rest (D) while the user stands, pivots, exercises, walks on or rests the injured leg. The invented ambulatory device in accordance with one embodiment of the invention is lightweight so that hand grip 20 or pressure grip 22 is all that is needed to easily stabilize, lift or otherwise maneuver the ambulatory device. Rubber tip 18 will be understood to provide traction against an ambulatory surface on which the user stands, exercises, walks or rests.

Two cushioned grips 20, 22 are provided, in accordance with one embodiment of the invention. Lower (hand) grip 20 provides a sure but comfortable hand grip at crotch level for manipulating vertical member 12 and for leveraging, positioning, aligning and securing lower limb support brace 14 between the lower limb of the user's leg and the ambulatory surface. In other words, lower (hand) grip 20 is used to hold onto invented device 10 and to maintain an upward force of lower limb, e.g. knee, support member 16 against the lower limb, e.g. the bent knee, while walking. Upper (pressure) grip 22 may be included for esthetics but it also serves a function: A user can squeeze it between the upper arm and the side of the chest (see FIG. 1B). This allows a user to carry an item such as a glass or mug or plate of food with either or both hands while walking for short distances using invented device 10. This ability to have both hands free during use is an extraordinary advantage of the invented device, and is a particularly novel feature of the invention.

Referring still collectively to FIGS. 2 and 3, it will be understood that lower limb support member 16 is mounted in accordance with one embodiment of the invention in axial alignment with the congruent long axes of an inner support tube 24 and an outer support tube 26 that form a part of lower limb support brace 14. (Thus, lower limb support member 16 includes a central mounting region affixed on inner support tube 24 of lower limb supporting brace 14, thereby ensuring proper axial load-bearing of the user's weight.) Those of skill in the art will appreciate that this ensures the bulk of the user's weight is borne in axial alignment by the inner and outer support tubes, which will be understood to be the primary load-bearing members of device 10. Those of skill in the art will also appreciate that inner support tube 24 ‘telescopes’ within outer support tube 26, or smoothly slides along their congruent axes, in accordance with one embodiment of the invention, to facilitate height adjustment of lower limb support member 16 relative to tip 18. A desired height C of lower limb support member 16 thus is fixed above the ambulation surface by sliding inner support tube 24 within outer support tube 26 and tightening a bolt/nut combination B3 that forms a part of a height-adjustment clamp 28.

Those of skill in the art will appreciate that any suitable lower limb support member height adjustment mechanism is contemplated as being within the spirit and scope of the invention. For example, an Allen-head or equivalent set screw may be used instead of a clamp and bolt assembly in the illustrated embodiment, for better security. Or the height of the support member can be adjusted by adjusting the height of a single-tube support brace relative to the vertical member, e.g. by providing more than two spaced-apart through holes in one of the vertical extents to yield a range of height-relative mounting options between the lower limb support brace and the vertical handle member.

It will be understood that the present invention is not limited to the method or detail of construction, fabrication, material, application or use described and illustrated herein. Indeed, any suitable variation of fabrication, use, or application is contemplated as an alternative embodiment, and thus is within the spirit and scope, of the invention.

From the foregoing, those of skill in the art will appreciate that several advantages of the present invention include the following.

The present invention provides means for more freedom in mobility, avoidance of damage to the user's wrist/hand and armpit areas; and exercise of the upper half of the injured leg that overcomes the deficiencies of known prior art devices. The invented device provides more freedom in mobility by allowing the use of one or both hands while walking (ambulating) with the device. The invented device provides a means of avoiding pain caused by nerve compression in the armpit area because none of the user's weight is supported by the armpit as is the case with conventional crutches. Similarly, wrist and hand pain are avoided because none of the user's weight is supported by the hands. The invented device inherently exercises the upper half of the injured leg because the user's normal body weight is supported by the knee and thigh of the injured leg.

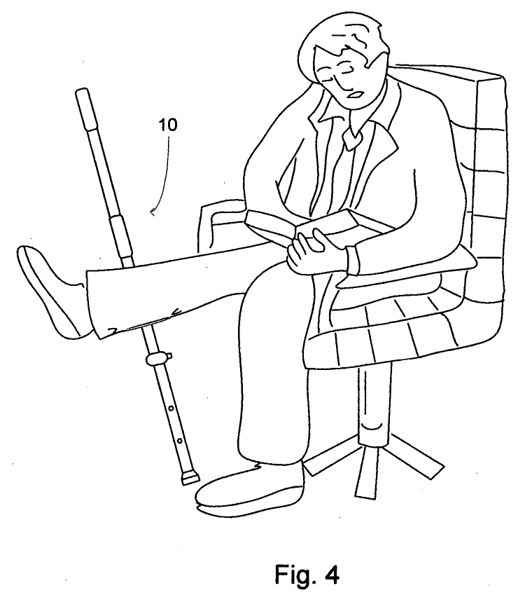

Moreover, from FIG. 4, it will also be understood that, in addition to providing the aforementioned advantages described above, the invented device can also be used as a foot rest to keep the injured leg elevated while the user is in a sitting position. Thus, a user of the invented device can use the device to elevate his or her injured leg while comfortably sitting down to rest. Those of skill in the art will appreciate that, by this method of use of the invention, cushioned lower limb support member 16 instead of serving to support the user's bent knee serves instead to comfortably support a rear lower region of the injured leg in a generally horizontal and extended configuration. It is generally known that injured limb extremities at rest heal more quickly when elevated relative to the heart due to increased circulation.

The invented device thus allows a patient with a lower leg injury (one that involves injury below the knee) to move about without the problems incurred by the use of conventional crutches that are used in pairs. This device allows one hand to be free at all times and both hands to be free for short periods or distances. This device is lightweight and easily can be picked up, placed under the knee of the injured leg and stabilized for standing, walking, exercising or resting. There are no straps or fittings to be dealt with and, because the device is not strapped to the user, it can be easily set aside when the user sits down or otherwise does not need the device. Because of its symmetrical design, the device can be used on either the left or right leg. Conventional crutches require the patient to place a great deal of weight and strain on both wrists and hands as well as a great deal of weight on the armpit areas in order to support the weight of the person using them. This unnecessary burden of weight and strain generally causes more problems for the patient, including nerve damage in the wrist\hand and armpit areas. In accordance with the present invention, all of the patient's weight is placed on the lower limb, e.g. the bent knee, of the injured leg. This distributes the weight evenly across both knees and upper legs in the same way a person with no leg injury would experience.

While it is lightweight, the invented device is very of durable construction. Its weight-bearing member, i.e. the lower limb support brace, is oriented substantially in axial alignment with the user's upper leg, and is manipulated into position and alignment therewith by an elongate, grippable handle that obtains a mechanical advantage through leverage and that allows gripping by an extended hand for one free-handed operation or between an arm and torso for two free-handed operation. Moreover, the invented device has adjustable dimensions in critical fit areas to provide a range of suitable fits for various user sizes and preferences.

It is further intended that any other embodiments of the present invention that result from any changes in application or method of use or operation, method of manufacture, shape, size, or material which are not specified within the detailed written description or illustrations contained herein yet are considered apparent or obvious to one skilled in the art are within the scope of the present invention.

Accordingly, while the present invention has been shown and described with reference to the foregoing embodiments of the invented apparatus, it will be apparent to those skilled in the art that other changes in form and detail may be made therein without departing from the spirit and scope of the invention as defined in the appended claims.

1. A lower limb-support ambulatory device comprising:
an elongate lower limb supporting brace including at an upper extreme a lower limb support member;
an elongate vertical stabilizing handle for stabilizing the lower limb supporting brace, the vertical stabilizing handle being securely grippable by a user without use of the armpit;
the lower limb supporting brace including at a lower extreme a tip configured to frictionally engage an ambulatory surface on which the user ambulates;
the lower limb supporting brace and the vertical stabilizing handle being joined to one another in a lower region of the stabilizing handle.

2. A lower limb-support ambulatory device comprising:
an elongate lower limb supporting brace including at an upper extreme a lower limb support member;
an elongate vertical stabilizing handle for stabilizing the lower limb supporting brace, the vertical stabilizing handle being securely grippable by a user without use of the armpit;
the lower limb supporting brace including at a lower extreme a tip configured to frictionally engage an ambulatory surface on which the user ambulates;
the lower limb supporting brace and the vertical stabilizing handle being affixed to one another along parallel extents thereof.

3. The device of claim 2, wherein the lower limb support member includes a central mounting region affixed on the lower limb supporting brace in substantial alignment with a long axis of the lower limb supporting brace.

4. The device of claim 3, wherein the lower limb supporting brace includes first and second elongate members configured to slide relative to one another along a central elongate axis of the first and second members with one of the first and second members positioned inside the other of the first and second members, and wherein one of the first and second members is affixed to the lower limb support member in the central mounting region thereof and wherein the first and second members are fixably adjustable relative to one another to adjust the height of the lower limb support member above the ambulatory surface.

5. The device of claim 4, wherein the stabilizing handle includes an elongate member dimensioned to extend generally vertically to a height approximately intermediate the user's waist and armpit.

6. The device of claim 5, wherein the elongate member of the stabilizing handle includes a lateral step in a lower region thereof, the step spacing apart a long axis of an upper gripping region of the elongate member from the central axis of the first and second members of the lower limb supporting brace.

7. The device of claim 6 which further comprises:
one or more hand grips extending along the upper gripping region Of the elongate member, the hand grips providing traction for a user's grip thereon.

8. The device of claim 7, wherein an outer surface of the upper gripping region of the elongate member is generally circular in cross section, and wherein the one or more hand grips include a generally circular hollow central region dimensioned to fixably slide along the outer surface of the upper gripping region of the elongate member.

9. The device of claim 8, wherein the lower limb support member includes a smoothly curved semicircular concavity in an upper surface thereof, a radius describing the semicircle extending along the user's lower limb.

10. The device of claim 9, wherein the one or more hand grips are two or more, at least one of which is positioned along the handle region approximately at the height of the user's crotch and at least another of which is positioned at the extreme upper end of the handle region, wherein the two or more hand grips are padded for comfort.

11. The device of claim, 10, wherein the lower limb support member in the upper surface thereof and at least partway across the concavity is padded for comfort.

12. The device of claim 11, wherein at least the elongate member of the lower limb stabilizing handle and the first and second members of the lower limb support brace are hollow generally circular tubes of a lightweight durable material taken from a group of materials including aluminum, titanium, graphite, fiberglass and plastic, alone or in combination.

13. A knee support ambulatory device comprising:
a vertical member configured to be grippable by a user in an upper region thereof;
a knee support brace affixed to the vertical member, the knee support brace configured at an upper extreme thereof with a bent-knee support member to support a bent knee of the user;
the knee support brace including at a lower extreme thereof a tip configured to frictionally engage an ambulation surface on which the user ambulates while gripping the vertical member with the bent knee reposing on the bent-knee support member.

14. The device of claim 13, wherein the bent-knee support member is height-adjustable relative to the tip.

15. The device of claim 13, wherein the vertical member is elongate and includes at least one cushioned gripping collar aligned with its long vertical axis.

16. The device of claim 15, wherein the gripping collar is slidably but securely height-adjustable.

17. The device of claim 13, wherein the vertical member is elongate and includes plural cushioned gripping collars aligned with its long vertical axis, the plural gripping collars being spaced apart along the upper region of the vertical member.

18. The device of claim 13, wherein the vertical member is hollow.

19. The device of claim 13, wherein the bent-knee support member is cushioned in an upper surface region thereof comfortably to support at least part of a body weight of the user on the bent knee thereof.

20. The device of claim 13, wherein the knee support brace is positioned laterally inwardly from the vertical support member when the device is in use.

21. The device of claim 13, wherein the bent-knee support member is height-adjustable relative to the tip and is cushioned in an upper surface region thereof comfortably to support at least part of a body weight of the user on the bent knee thereof, wherein the knee support brace is positioned laterally inwardly from the vertical member, and wherein the vertical member is hollow and elongate and includes at least one cushioned gripping collar aligned with its long vertical axis and slidably but securely height-adjustable therealong.

22. The device of claim 13, wherein the vertical member is elongate and generally circular in cross section and is dimensioned relative to the user such that the upper extreme of the vertical member reaches a height on the user approximately intermediate the waist and armpit thereof.
